(19) United States
(12) Patent Application Publication
(10) Pub. No.: US 2009/0229342 A1
(43) Pub. Date: Sep. 17, 2009

(54) HYBRID PLASMON DAMPING SENSOR

(76) Inventor: ALI KAAN KALKAN, Stillwater, OK (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(21) Appl. No.: 12/347,771

(22) Filed: Dec. 31, 2008

Related U.S. Application Data
(60) Provisional application No. 61/018,222, filed on Dec. 31, 2007.

(51) Int. Cl. G01N 33/00; G01J 3/42

(52) U.S. Cl. 73/23.2; 73/53.01; 356/319; 977/773; 977/810

(57) ABSTRACT

A system and method for measuring an agent in an environment is disclosed. The method includes providing a substrate, coating the substrate with noble metallic nanoparticles, exposing the coated substrate to the environment, and determining the existence of the agent from variation in the hybrid plasmon extinction peak of the metallic nanoparticles.

### CROSS REFERENCE TO RELATED APPLICATIONS

This application claims the benefit of and priority to U.S. Provisional Patent Application No. 61/018,222 entitled “HYBRID PLASMON DAMPING SENSOR,” filed Dec. 31, 2007, the contents of which are hereby incorporated by reference.

### FIELD OF THE INVENTION

This disclosure is related to trace level sensors in general and, more specifically, to plasmon sensors.

### BACKGROUND OF THE INVENTION

Since the medieval centuries, noble metal nanoparticles have provided the unfading brilliant colors in stained glass windows, pottery, and paintings. It was not until Gustav Mie (1908), however, these beautiful colors were attributed to the resonant coupling of light with the collective electron oscillations of nanoparticles, namely the localized surface plasmon modes. This fascinating phenomenon, that is the resonant coupling of light with localized surface plasmons, is subject to spectral shifts when the nanoparticles undergo electromagnetic or charge interactions with their environment. Such spectral shifts are easily detectable by conventional spectrometers, and at times with the naked eye. As a result, localized surface plasmon resonance (LSPR) opens the door to the development of a new generation of chemical sensors.

With the launch of intense research activity in the fabrication and utilization of nanostructures in recent years, localized surface plasmon resonance (LSPR) sensors employing metal nanoparticles gained significant attention with the objective of detecting biomolecules, explosives, toxins, and warfare agents at trace levels. Up to present, LSPR sensing demonstrations exploited the frequency and intensity shift of the LSPR optical extinction peak due to the variation in: 1) polarizability (i.e., refractive index) of the medium surrounding the metal nanoparticle; and, 2) charge transfer to/from the metal nanoparticle.

The variation in the LSPR frequency induced by changes in refractive index of the near ambient of the nanoparticle has been exploited as a sensing mechanism by a number of research groups. Amanda J. Haes and coworkers [A. J. Haes, W. P. Hall, L. Chang, W. L. Klein, and R. P. V. Duyne, “A Localized surface plasmon resonance biosensor: first steps toward an assay for Alzheimer's disease,” Nano Letters, vol. 4, pp. 1029-1034, 2004. A. J. Haes and R. P. Van Duyne, “A nanoscale optical biosensor: sensitivity and selectivity of an approach based on the localized surface plasmon resonance spectroscopy of triangular silver nanoparticles,” J. Am. Chem. Soc., vol. 124, pp. 10596-10604, 2002. Z. Jing, Z. Xiaoyu, Y. C. Ranjit, A. J. Haes, and R. P. Van Duyne, “Localized surface plasmon resonance biosensors,” Nanomedicine, Vol. 1, pp. 219-228, 2006] used the frequency shift in the plasmon mode of the silver nanotriangles as LSPR sensor for detection of Alzheimer disease. The lower frequency shift in the optical extinction of the silver nanotriangles was used to determine the interaction of amyloid 1-derived diffusible ligands (ADDL) and anti-ADDLs which are involved in development of Alzheimer's disease. They confirmed that the plasmon frequency of the silver nanotriangles shifts towards red with increasing density and thickness of the adsorbate layers (ADDL and anti-ADDLs). Okamoto et al., demonstrated the variation of the optical frequency and intensity of the gold nanoparticle monolayers with refractive index of the surrounding medium [T. Okamoto, I. Yamaguchi, and T. Kobayashi, “Local plasmon sensor with gold colloidal monolayers deposited upon glass substrates,” Optics Letters, vol. 25, pp. 372-374, 2000]. They concluded that the optical extinction of the monolayers of the gold nanoparticles shift towards red, when the refractive indices of the immersed liquids were increased. They also noticed that the intensity of the optical extinction of gold nanoparticles was increased with increase in the refractive index. Mock and coworkers demonstrated red shift in the spectrum of the individual silver nanoparticles with increase in the refractive index of oil surrounding the metal nanoparticles [J. J. Mock, D. R. Smith, and S. Schultz, “Local refractive index dependence of plasmon resonance spectra from individual nanoparticles,” Nano Letters, vol. 3, pp. 485-490, 2003].

Another cause for variation in the frequency and the intensity of the LSPR is electron transfer between the adsorbate and nanoparticle. The direction of transfer depends on the Fermi level or electronegativity difference between the adsorbate and the metal nanoparticle. When electron transfer occurs towards the metal nanoparticle, the surface plasmon resonance frequency shifts towards higher frequencies, and vice versa. [A. Henglein and M. Giersig, “Optical and chemical observations on gold-mercury nanoparticles in aqueous solution,” J. Phys. Chem. B. vol. 104, pp. 5056-5060, 2000. T. Morris, H. Copeland, E. McLinden, S. Wilson, and G. Szulczewski, “The effect of Mercury adsorption on the optical response of selected gold and silver nanoparticles,” Langmuir, vol. 18, pp. 7261-7264, 2002. T. Morris, K. Kloepper, S. Wilson, and G. Szulczewski, “A spectroscopic study of mercury vapor adsorption on gold nanoparticle films,” J. Col. Int. Sci., vol. 254, pp. 49-55, 2002.]. In addition, the intensity of the optical extinction increases with increase in the number of conduction electrons. Henglein et al. [Reference cited above] during their study on bimetallic colloids observed surface plasmon frequency shifts towards higher frequency in the gold and silver nanoparticles, when the mercury was introduced in the nanoparticle colloidal solution. Morris et al. [Reference cited above] demonstrated that the optical extinction of dipolar plasmon mode of the silver nanoparticles blue shift more than gold nanoparticles in response to mercury vapor. They also showed that the smaller particles exhibit more blue shift than larger particles.

What is needed is a method for addressing the shortcomings of the methodologies described above.

### SUMMARY OF THE INVENTION

The invention disclosed and claimed herein, in one aspect thereof, comprises a method for measuring the concentration of an agent in an environment. The method includes providing a substrate, coating the substrate with noble metallic nanoparticles, exposing the coated substrate to the environment, and determining the existence of the agent from variation in the hybrid plasmon extinction peak of the metallic nanoparticles. In another embodiment, the method includes determining by spectrophotometry from a variation in a damping factor of the hybrid resonance of the metallic nanoparticles whether the substrate has been exposed to the agent.

The coated surface may be annealed, such as by heating the coated substrate to about 300 degrees Celsius for about 1 minute. Coating the substrate with noble metallic nanoparticles may further comprise providing a monolayer of metallic nanoparticles on the substrate and/or functionalizing the metallic nanopoparticles to provide selective detection of the agent.

The substrate may comprise a semiconductor substrate, such as silicon. The substrate could also be a silicon on glass covered substrate. Coating the substrate with noble metallic nanoparticles may include immersing a semiconductor film in a metal salt solution.

Determining the existence of the agent from variation in the hybrid plasmon extinction peak of the metallic nanoparticles may include determining a width of a hybrid plasmon resonance peak, and/or determining an intensity of the hybrid plasmon resonance peak. The amount of the agent may also be determined from the hybrid plasmon extinction peak.

The invention disclosed and claimed herein, in another aspect thereof, comprises a sensor for measuring concentration of an agent. The sensor includes a substrate, a monolayer of noble metallic nanoparticles on the substrate, and means for detecting the presence and concentration of the agent by measuring variation in the hybrid plasmon extinction peak of the metallic nanoparticles. The substrate may be a semiconductor film on glass. The monolayer of noble metallic nanoparticles on the substrate may be annealed.

### BRIEF DESCRIPTION OF THE DRAWINGS

FIG. 1: Schematics illustrating the hybridization of two dipolar plasmon modes.

FIG. 2: Representative atomic force microscopy height images of the Ag nanoparticles after electroless reduction on Si films for different film immersion times: (a) 5 seconds; (b) 10 seconds; (c) 20 seconds.

FIG. 3: AFM surface images of the 10-second-immersion sample as prepared (a), and after annealing (b).

FIG. 4: Hybrid plasmon extinction spectra before and after annealing.

FIG. 5: Optical extinction spectra of the silver nanoparticles annealed on the hot plate at 300° C. for different time intervals.

FIG. 6: Time series optical extinction spectra for Ag nanoparticles being exposed to Hg vapor.

FIG. 7: Kinetics of damping (a) and peak height (b) of hybrid plasmon extinction of the silver nanoparticles exposed to mercury in air. (c) The number of mercury adsorbates as a function of time. (d) Variation of damping with number of adsorbates.

FIG. 8: Time evolution of the hybrid plasmon extinction in response to 25 ppb H₂S (10-s-immersion sample after anneal).

FIG. 9: Picture showing: (a) the optical cell with sensor substrate immobilized inside; (b) injection of mercury into the optical cell, which is placed in the cuvette holder while the optical extinction measurement is in progress.

### DETAILED DESCRIPTION OF THE PREFERRED EMBODIMENTS

The present disclosure describes a new sensing technology that is based upon changes in localized surface plasmon resonance (LSPR). In one embodiment, this is the increase in damping of the hybrid plasmon mode due to molecular adsorption at the surface of the plasmonic nanostructures (e.g., nanoparticles). This disclosure teaches methods to obtain and use this new mode of sensing.

Up to present, all LSPR sensing demonstrations exploited the frequency and intensity shift of the LSPR optical extinction peak due to the variation in: 1) polarizability (i.e., refractive index) of the medium surrounding the metal nanoparticle; and, 2) charge transfer to/from the metal nanoparticle. On the other hand, the present disclosure brings into play a third impetus that will induce a change in LSPR. This is the increase in damping of the hybrid plasmon mode due to molecular adsorption at the surface of the nanoparticle. Hybrid plasmon modes develop when strong electromagnetic coupling between adjacent nanoparticles occur. “Plasmon hybridization” may be interpreted as interference or superposition of regular plasmon modes (modes seen for isolated particles).

The phenomenon of “plasmon hybridization” is analogous to the formation of molecular orbitals from the combination of atomic orbitals in covalent bonding. Hybrid plasmon modes exhibit higher and faster spectral shifts in response to molecular adsorption. Therefore, they offer a higher sensitivity and a shorter response time than regular plasmon modes (modes seen for isolated particles) do.

Various embodiments of the hybrid plasmon damping sensors of the present disclosure reports the width (damping factor) and intensity of the hybrid plasmon resonance associated with a monolayer of noble metal nanoparticles. The present disclosure employs two approaches for the determination of the concentration of the agent being detected. In the first embodiment, the two parameters said above (width and intensity of the hybrid plasmon resonance peak), continuously measured by optical extinction, are substituted in a theoretical relation to quantify the number of electrons gained or lost (by the hybrid plasmon) due to the adsorbed molecules or atoms. The change in the number of free electrons precisely equals the number of adsorbates. The agent concentration is derived from the kinetics of number of adsorbates. In the second embodiment, only damping factor (width of the hybrid plasmon resonance) is utilized. The concentration is derived from the kinetics of damping factor.

The concept of plasmon damping may be explained using an oscillator model, where a spring-mass system is resonantly excited with a harmonically acting force. The harmonic force corresponds to the electromagnetic field (light). The natural frequency of the system corresponds to the plasmon frequency. Furthermore, the viscous friction, or damping, of the system is analogous to the electron scattering at the nanoparticle surface. The increase of the viscous dissipation (damping) will amount to reduced amplitude of the oscillation. In the nanoparticle, this happens when a molecule adsorbs on the nanoparticle surface and increases the electron scattering. The consequence is reduction in optical density of the plasmon mode. This is measured as a reduction in optical extinction. Accordingly, the present disclosure contemplates the damping of plasmon modes as a probe to detect adsorbates on the nanoparticle surface. More specifically, these are the hybrid plasmon modes. The present disclosure demonstrates that this sensing mechanism offers superior sensitivity over conventional LSPR sensing approaches.

LSPR sensors may make use of gold and silver nanoparticles due to the occurrence of LSPR in the visible region of electromagnetic spectrum in these metals. Among all metals, silver exhibits the strongest LSPR due to its lowest plasmon damping. Accordingly, the hybrid plasmon damping sensor of the present invention can be based on gold (Au) or silver (Ag) nanoparticles. For example, Au or Ag nanoparticles may be used for detecting traces of sulfur compounds in H₂ (e.g., in fuel cell applications), using the methods of this disclosure. This is because Ag and Au nanoparticle surfaces have the highest affinity for sulfur. Likewise, both Ag and Au have high affinity for Hg. Unlike sulfur (S), mercury (Hg) shifts the hybrid plasmon peak in the direction of increasing wavelength. Therefore, a hybrid plasmon damping sensor based on Au or Ag can distinguish between S and Hg.

If both S and Hg are in the medium, then selectivity for either S or Hg may be needed. One way of achieving this is to use a self-assembling molecular monolayer (SAM) that is permeable either to S or Hg. On the other hand, for species having low affinity for Au and Ag, the nanoparticle surfaces need to be chemically functionalized for specificity. SAMs may be used for functionalization of Au and Ag surfaces with organothiols for tailoring the surface chemistry. Therefore, the hybrid plasmon damping sensor of this disclosure can be made specific to a large number of molecules.

In conventional surface plasmon resonance sensing applications, the nanoparticles are functionalized with specific molecules, which have high affinity to the agent to be detected. In an LSPR biosensor targeting prostate cancer antigen for example, the nanoparticles are functionalized with specific antibodies recognizing the antigen. In other words, those antibodies have the highest affinity to the prostate cancer antigens.

In the case of detecting S or Hg, however, both Ag and Au already have the highest affinity for S or Hg, respectively. Therefore, functionalization of the nanoparticles is not needed. Strong interaction of S or Hg with Au and Ag distorts the local electric potential at the metal surface leading to increased electron scattering. In case the intrinsic scattering is minimal, this additional scattering will have a prominent impact on hybrid plasmon damping. The metal nanoparticles will be treated as described in the present disclosure for the minimization of intrinsic scattering or damping.

The present disclosure also teaches practices for obtaining a well-resolved, intense, and sharp hybrid plasmon extinction peak for higher sensitivity. The disclosure demonstrates that “nanometal-on-semiconductor” approach offers these attributes. In one embodiment, electroless reduction of metal nanoparticles on silicon is utilized. This enables the fabrication of monolayer of surfactant-free, size-controlled metal nanoparticles on silicon. During the synthesis, the interparticle separation is observed to shrink to as low as a few nanometers, but the particles never impinge each other. As a result, strong and well-resolved hybrid plasmon modes develop enhancing the sensitivity. This has been attributed to the unique “nanometal on semiconductor” approach, wherein the charge transfer between the metal nanoparticles and silicon film gives rise to Coulombic repulsion between the particles.

Another aspect of the various embodiments of the present disclosure is the dramatic reduction of intrinsic hybrid plasmon damping by annealing of the nanoparticle monolayers. The minimization of intrinsic plasmon damping provides a substantial enhancement in sensitivity. Once the intrinsic damping is minimized, damping due to molecular adsorption on the nanoparticle causes a more significant fractional change in total damping.

1. Optical Extinction

The present disclosure employs hybrid plasmon modes of the nanoparticles as the sensing probe. These modes interact with light in two ways: absorption of light and scattering of light. The sum of these two components is called the “optical extinction”, which is measurable. Accordingly, a brief review optical extinction is given here.

In optical spectroscopy, extinction, E, is defined as E=−log(T), where T is the transmission, which is easy to measure using a spectrophotometer. When a light beam is blocked by a layer of nanoparticles, part of the light won't interact with the nanoparticles and be transmitted through (transmission). However, part of the light will be absorbed and scattered by the nanoparticles. In spectroscopy, the absorbed or scattered photons (light) are called the “extinct” photons (light) in the sense that they will not reach the detector in a simple transmission setup. In a medium where photons get extinct, the intensity of light decays exponentially in the propagation direction and the transmitted light will be: I=Iₒexp(−αd), where I, Iₒ and d are the transmitted and incident light intensities, and the distance traveled by light, respectively. The constant ox is the “extinction coefficient”, and is a measure of how intense the medium absorbs+scatters the light. The exponential decay is simply the solution of differential equation that states; “rate of amount of light lost (absorbed+scattered) is proportional to its intensity” (Beer Lambert Law). Therefore, the extinction coefficient can simply be calculated from αd=−log(I/Iₒ), where (I/Iₒ) is defined as the transmission, and ad is defined as the extinction.

Optical spectrometers are used to measure the transmission and extinction. In general, an optical spectrometer consists of a light source, from which the light is incident on the sample placed inside a cuvette holder. The intensity of the transmitted light is measured using a light detector.

2. Hybrid Plasmon Modes

An isolated Ag nanoparticle, smaller than 30 nm, exhibits dipolar surface plasmon resonance, which occurs at the frequency ω_{d}=ωₚ/<3 in free space, where ωₚ is the bulk plasmon frequency, which is given by

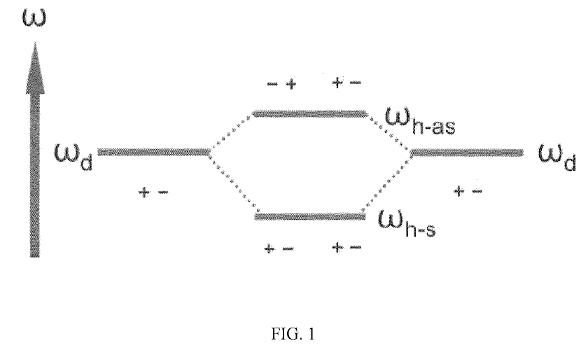

ωₚ=√(4πe²Nₑ/mₑ), (2.1)

where e, Nₑ, and mₑ are the electron charge, density and mass [U. Kreibig and M. Vollmer, Optical Properties of Metal Cluster: Springer, New York, 1995]. On the other hand, when two nanoparticles are brought close such that the interparticle separation is less than the particle diameter, the individual plasmon modes start to interfere and form two hybrid combinations: the symmetric mode constructed from in-phase dipole oscillations (ωₕ₋ₛ<ω_{d}); and antisymmetric mode constructed from out-of-phase dipole oscillations (ωₕ₋ₐₛ>ω_{d}), as illustrated in FIG. 1. Because the net dipole moment of the antisymmetric combination is zero for identical spheres, the antisymmetric modes are not easily excited by light and observed (i.e., dark plasmons), in contrast to the symmetric hybrid plasmons (bright plasmons).

3. Plasmon Damping

In quantum mechanical terms, when a photon (particle of light) couples with coherent oscillation of conduction electrons, a plasmon is created. Plasmon is a very short-lived quasi-particle. It lives for tens of femtoseconds at most, and decays back to a photon or phonons (heat). Decay of the plasmon to a photon occurs in terms of light scattering. On the other hand, the decay of plasmon to phonon(s) is called absorption. Extinction, as defined previously, is the sum of scattering and absorption: Extinction=Scattering+Absorbtion.

Within the context of the model mass-spring-damper system, extinction is the dissipated power that is balanced by the power nanoparticle extracts from the electromagnetic field. The power extracted from the field, is either dissipated to heat (absorption) or radiated back to the field (scattering).

From the point of power dissipation, plasmon extinction is caused by plasmon damping, which is characterized by Γ: the damping constant. Exploiting the Mathiessen's Rule, Γ can be split into scattering and absorption components as: Γ=Γₛ+Γₐ

In this respect, plasmon decay or damping occurs either radiatively (scattering) or non-radiatively (absorption). As mentioned above, plasmon is a short-lived state with a time constant of ˜10 fs. In simple terms, plasmon is a very “fragile” quasi particle whose decay or damping is very easy. Namely, any effect that disturbs the cohesive motion of “many” electrons contributing to the plasmon collapses the plasmon. In particular, radiative damping occurs when nanoparticle size is not sufficiently small compared to the wavelength. In this case, the phase of the electric field is not uniform throughout the nanoparticle, so is the phase of the electron motion. As a result, some electrons cannot follow the others in phase and get “retarded”. Therefore, the plasmon collapses and releases its energy in terms of a photon. This is radiative plasmon damping, accounting for light scattering. Indeed, gold and silver nanoparticles larger than 30 nm, experience significant radiative damping, which allow these particles be seen under optical microscope in dark field.

Non-radiative damping (absorption), on the other hand, occurs through a diversity of pathways. The most common mechanism for non-radiative damping is the scattering of electrons from defects and nanoparticle surface. Again, this scattering perturbs the cohesive electron motion. Though, the plasmon transfers its energy to an electron-hole pair instead of a photon. In turn, the energy of the electron-hole pair thermalizes to phonons (heat). Non-radiative damping can also take place through “chemical interface damping”, wherein the cohesive motion of the plasmon is perturbed by the trapping of some electrons in the adsorbate-induced quantum states. The adsorbate-induced states simply form by chemical bonding of guest atoms or molecules (from the environment) on the nanoparticle surface. The temporary “leakage” or “trapping” of the conduction electrons in these states dephases the plasmon and causes it to collapse. Again, the energy is thermalizes to phonons (heat).

In the operation of hybrid plasmon damping sensor of the present invention, the sensing mechanism is not limited to chemical interface damping, but also includes electron scattering and radiative damping. Electron scattering is simply caused by the perturbation of the electric potential at the surface of the nanoparticle by guest atoms or molecules (adsorbates).

4. Quantification of Number of Adsorbates

In 1908, by fully solving for the Maxwell's Equations, Mie obtained an analytical solution for the extinction cross section of a solid sphere, σₑₓₜ, in an electromagnetic field of angular frequency ω. In case the sphere's diameter is significantly smaller than the wavelength of light (e.g., 20 times smaller), σₑₓₜ is given by

$${σ}_{ext} ( ω ) = 9 \frac{ω}{c} {ɛ}_{m}^{3 / 2} V ( \frac{{ɛ}_{2} ( ω )}{{[ {ɛ}_{1} ( ω ) + 2 {ɛ}_{m} ]}^{2} + {{ɛ}_{2} ( ω )}^{2}} ) \qquad ( 4.1 )$$

where Σ₁(ω)+i∈₂(ω) and ∈ₘ are the dielectric functions for the sphere and the surrounding medium, respectively [Kreibig and Vollmer]. V is the sphere volume, and c is the velocity of light. σₑₓₜ is defined such that, for a single nanoparticle, P_{extinction}=Iσₑₓₜ, where P_{extinction} is the electromagnetic power scattered and absorbed (extinction) by the nanoparticle, and I is the incident irradiation or the light intensity (power per unit area).

Clearly, σₑₓₜ has a resonance for ∈₁(ω)=−2∈ₘ, at ω=ωₒ, that is known as the localized surface plasmon resonance (dipolar LSPR), while ω=ωₒ is called the plasmon frequency. Obviously, ω=ωₒ will shift with the variation in ∈ₘ since ωₒ=∈₁⁻¹(−2∈ₘ). This forms the basis of detection for prior LSPR sensors. Alternatively, σₑₓₜ is subject to changes due to the changes in ∈₂(ω) that is the basis for the detection technique of the present disclosure.

The dielectric function for the metals can be approximated from free electron theory as

$$\begin{matrix}{ɛ}_{1} ( ω ) = 1 - \frac{{ω}_{p}^{2}}{{ω}^{2}} \\ and & ( 4.2 a ) \\ {ɛ}_{2} ( ω ) = \frac{{ω}_{p}^{2}}{{ω}^{3}} Γ & ( 4.2 b )\end{matrix}$$

where ωₚ is the bulk plasmon frequency as introduced earlier and Γ is the phenomenological damping rate [Krebig and Vollmer]. In vacuum and gases ∈ₘ≅1, from which

$${ω}_{o} = \frac{{ω}_{p}}{\sqrt{3}} , \\ {ɛ}_{1} ( ω ) = \frac{( {ω}^{2} - 3 {ω}_{o}^{2} )}{{ω}^{2}} , \\ and \\ [ {ɛ}_{1} ( ω ) + 2 ] = \frac{3 ( {ω}^{2} - {ω}_{o}^{2} )}{{ω}^{2}} .$$

Using these relations and noting that (ω²−ωₒ²)²≅4ωₒ²(ω−ωₒ)² at the vicinity of resonance, the expression for σₑₓₜ reduces to a Lorentzian:

$${σ}_{ext} = \frac{3}{4} ( V / c ) {ɛ}_{m}^{3 / 2} \frac{{ω}_{o}^{2} Γ}{{( ω - {ω}_{o} )}^{2} + {( Γ / 2 )}^{2}} \qquad ( 4.3 )$$

Next, the number of adsorbates will be derived from Equation. 4.3. At resonance

$$( ω = {ω}_{o} ) , {σ}_{ext} = {σ}_{ext , peak} = ( 3 V {ɛ}_{m}^{3 / 2} / c ) \frac{{ω}_{o}^{2}}{Γ} .$$

### Remembering

$${ω}_{o} = \frac{{ω}_{p}}{\sqrt{3}}$$

and using the fact that plasmon frequency scales with the square of the electron density,

$$\begin{matrix}{ω}_{p} = \sqrt{( \frac{4 π {e}^{2} {N}_{e}}{{m}_{e}} )} & ( 2.1 ) \\ {σ}_{ext , peak} = ( \frac{4 π {e}^{2} V {ɛ}_{m}^{3 / 2}}{c {m}_{e}} ) \frac{{N}_{e}}{Γ} & ( 4.4 )\end{matrix}$$

When a molecule adsorbs on the nanoparticle, it causes a change in both Nₑ and Γ from Nₑₒ to Nₑₒ+ΔNₑ and from Γₒ to Γₒ+ΔΓ. Hence,

$${σ}_{ext , peak} ∝ \frac{{N}_{eo} + Δ {N}_{e}}{{Γ}_{o} + Δ Γ} \qquad ( 4.5 )$$

As discussed earlier, ΔΓ results from local distortion of the surface electric potential by the adsorbate or chemical interface damping, or increased radiative damping.

The impact of adsorption on Nₑ depends on the type of bond established. 1) Positive ΔNₑ. This occurs when the adsorbate contributes to the plasmon mode with free electron(s). Typically, metallic bonds account for this. As an example, mercury has high affinity for silver, and establishes a metallic bond contributing an electron to the conduction electron gas. Since this electron becomes completely delocalized in the silver nanoparticle, ΔNₑ is the number of mercury atoms adsorbed; i.e., ΔNₑ=N_{Hg}; 2) Negative ΔNₑ. This occurs when covalent bond(s) are established. Since, silver shares electron(s) with the adsorbate and these electrons are strongly localized in the covalent bond(s), the number of free electrons associated with the plasmon is reduced. A good example of this is the adsorption of H₂S on silver. This involves the chemical reaction: 2Ag+H₂S→Ag₂S+H₂. For each sulfur chemisorbed on the silver surface, 2 free electrons from silver metal are stolen to 2 Ag—S bonds. In other words: ΔNₑ=−N_{Ag-S}=−2N_{S}; 3) ΔNₑ≅0 (Physisorption). Both Case (1) and Case (2) are associated with chemisorption, where electron sharing takes place between the nanoparticle and the adsorbate.

Physisorption is due to Vander Waals or dipole—induced dipole bonds, which do not involve electron sharing or transfer. Therefore, physisorption can at most change the local polarizability or refractive index around the particle, leading to a shift in ωₒ. However, a change in Nₑ is not expected. Further, physisorption can perturb the electric potential at the nanoparticle surface dephasing the plasmon. Hence, physisorption is detectable from increase of damping: ΔΓ.

In the absence of heterogenous broadening of the plasmon resonance, extinction equals σₑₓₜ multiplied with a constant. Therefore, it follows from Equation. 4.5 that

$$H = C \frac{{N}_{eo} + Δ {N}_{e}}{Γ} \qquad ( 4.6 )$$

where H is the extinction peak intensity and C is the scaling factor. Therefore,

CΔNₑ=HΓ−CNₑₒ

$${H}_{o} = C \frac{{N}_{eo}}{{Γ}_{o}}$$

If is the intrinsic (in the absence of adsorbates) extinction peak intensity, then CNₑₒ=HₒΓₒ. Accordingly, ΔNₑ=(HΓ−HₒΓₒ)/C. Eliminating C,

$$Δ {N}_{e} = {N}_{eo} ( \frac{H Γ}{{H}_{o} {Γ}_{o}} - 1 ) \qquad ( 4.7 )$$

As discussed above, for the chemisorption of mercury on silver, we have ΔNₑ=N_{Hg}. Therefore,

$$Δ {N}_{Hg} = {N}_{eo} ( \frac{H Γ}{{H}_{o} {Γ}_{o}} - 1 ) \qquad ( 4.8 )$$

where N_{Hg} stands for the number of mercury atoms chemisorbed, while Nₑₒ equals the total number of intrinsic electrons contributing to the plasmon regardless of plasmon being a regular dipolar plasmon or hybrid plasmon. In the case of hybrid plasmon, it can be difficult to guess how many interacting nanoparticles contribute to the plasmon. However, this quantity can be determined during sensor calibration.

In summary, the method developed here calculates the number of adsorbates simply from the product of HΓ which can easily be extracted from the plasmon extinction spectrum: (peak intensity)×(full width at half maximum). Because the above expression derives directly from theory, no artificial fitting parameters are needed. Although, the derivation above is essentially performed for a single isolated nanoparticle, the hybrid plasmon resonance is also of Lorentzian character and characterized with certain values of ωₒ, Γ, H, and Nₑ. Therefore, Equations 4.7 and 4.8 apply to hybrid surface plasmon resonance, too. The variation in the damping due to adsorbates can be determined from optical extinction if the intrinsic plasmon damping is sufficiently reduced. When the intrinsic damping is reduced significantly, damping due to molecular adsorption becomes prominent and causes a subsequent decrease in hybrid plasmon extinction.

Normally, when nanoparticle synthesis involves reduction chemistry or vapor deposition at room temperature, the intrinsic damping, Γₒ is significantly large that, additional damping created by adsorbtion does not change the denominator to a discernible fraction. The present disclosure teaches that once the intrinsic damping, Γₒ, on the surface of a nanoparticle is reduced significantly, additional damping due to adsorption becomes the dominant mechanism for sensing. The intrinsic damping Γₒ arises from electron scattering due to various mechanisms: electron-electron scattering, electron-phonon scattering, electron-defect/surface scattering. Among these, only the last contribution can be reduced by processing. In particular, the present disclosure will teach Γ₀ can be reduced significantly by a rapid low-temperature annealing step.

Measurement of Concentration

The next step is the calculation of concentration from the kinetics. The adsorption of impurities on silver nanoparticles obeys the Langmuir's adsorption equation:

$$Δ {N}_{Hg} ( t ) = \frac{{Ck}_{a} N}{{Ck}_{a} + {K}_{d}} ( 1 - {e}^{- ( {K}_{a} + {K}_{d} ) t} ) ,$$

where N is the total number of adsorption sites. C is the concentration. Kₐ=Ckₐ and K_{d} are adsorption and desorption coefficients, respectively. As usual, ΔN_{Hg} is deduced from the peak intensity and width (damping) of the hybrid plasmon extinction.

By differentiating the Langmuir isotherm at t=0, we get

$$\frac{d}{d t} Δ {N}_{Hg} ( 0 ) = {Ck}_{a} N \qquad ( 4.1 )$$

Therefore, once kₐN is extracted from a single sensor calibration measurement, C can be computed from the slope of the Langmuir isotherm at t=0 as suggested by Equation 4.1.

C can also be deduced from the saturation regime at which

$${N}_{Hg} ( ∞ ) = \frac{{k}_{a} CN}{{k}_{a} C + {K}_{d}} = N \frac{C}{C + {K}_{d} / {k}_{a}} .$$

Accordingly, C can be evaluated as:

$$C = \frac{{N}_{Hg} ( ∞ ) ( {K}_{d} / {k}_{a} )}{N - {N}_{Hg} ( ∞ )} \qquad ( 4.2 )$$

Here, the values of N and K_{d}/kₐ can be determined by two sensor calibration measurements.

5. Synthesis of Noble Nanoparticles

In one embodiment, synthesis of noble nanoparticles involves synthesis of Ag or Au nanoparticles on a semiconductor film by the immersion of semiconductor film in metal salt solutions. The semiconductor may comprise Si, Ge, and III-V or II-VI compounds like GaAs, CdSe, CdS, InP.

Unlike conventional noble metal nanoparticle reduction techniques, the present approach does not require the use of a surfactant or capping agent for size and aggregation control of the nanoparticles, respectively. The absence of such agents on the nanoparticle surfaces is a substantial benefit in sensing. This minimizes the instrinsic damping and enables direct exposure of the nanoparticle surface to the ambient, therefore faster response and higher sensitivity. In addition, the chemistry of the surfactant-free Au/Ag surfaces can be easily modified with self-assembling monolayers as selective filters for sulfur compounds, mercury, iodine, or chlorine. Smaller or larger nanoparticle size can be achieved in a range from 20 to 100 nm by shorter immersion times or higher ion concentrations, respectively. The average spacing between the particles can also be tailored to significantly smaller than the particle size, such that a strong electromagnetic interaction between the particles induces hybridization of the individual surface plasmon modes.

In addition to serving as a reducer, the semiconductor film immobilizes the metal nanoparticles without the need for a linking agent. This immobilization of the nanoparticles has been attributed to the unique “nanometal on semiconductor” approach, wherein the charge transfer between the metal nanoparticles and silicon film gives rise to Coulombic attraction between the particles and the semiconductor substrate. The thin film approach of the present disclosure enables the transfer of the sensing activity onto any surface (e.g., glass, plastics), namely by Si thin film deposition and subsequent exposure to metal salt solutions. Therefore, the sensing can be integrated easily with other analytical techniques, and micro/nanofluidics. This enhances the extensibility and applicability of the developed sensor to various operational situations. For example, the interior surface of a gas chromatography column at its exit can be decorated with Ag or Au nanoparticles by the nanofabrication technique of the present disclosure after deposition of the Si film inside the gas column via low pressure chemical vapor deposition (LPCVD). This will enable sensing of the separated gas segments inside the column.

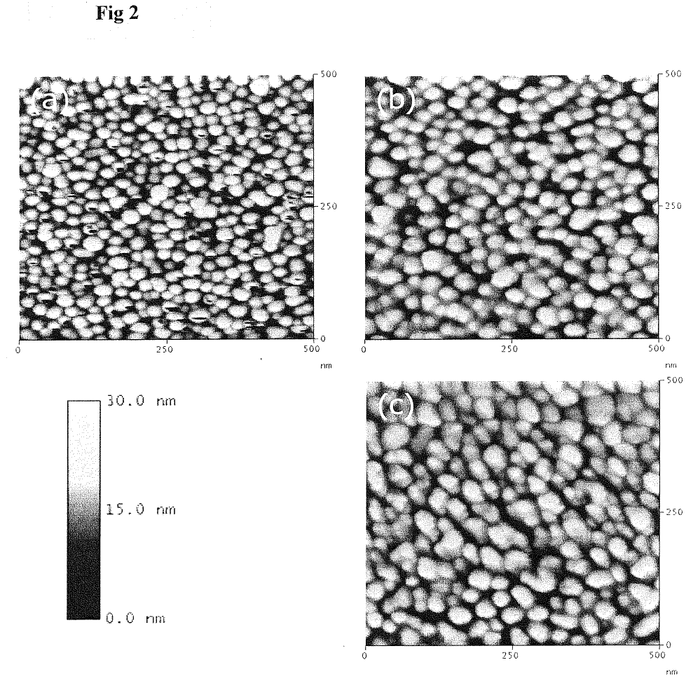

A typical Ag nanoparticle synthesis step of the present invention involves the immersion of a Si film deposited on glass into a 0.002 M AgNO₃ solution containing 0.1% HF for 10 s, where the Si film both serves as a reducer, and provides immobilization of the silver nanoparticles. The reason for including HF is to etch silicon oxide formed during the redox reaction. The Si film can be deposited by physical vapor deposition (PVD), low pressure chemical vapor deposition (LPCVD), or plasma-enhanced chemical vapor deposition (PECVD). For optical extinction measurements, the deposited substrates may be cut into smaller specimens of ˜5 mm×10 mm. Subsequently, the samples are immersed in AgNO₃ and then dipped in de-ionized water to stop the redox reaction almost instantaneously. The representative size and dispersion of the nanoparticles are shown in FIG. 2. as determined using atomic force microscopy (AFM). Obviously, the nanoparticle size is controllable with immersion time. For the sensor demonstrations disclosed in the present invention, 100 nm thick hydrogenated amorphous Si films deposited by PECVD were employed. An immersion time of 10 s was adopted. The nanoparticle synthesis was conducted in 0.002 M AgNO₃ and 0.1% HF.

6. Annealing and Minimization of Intrinsic Damping

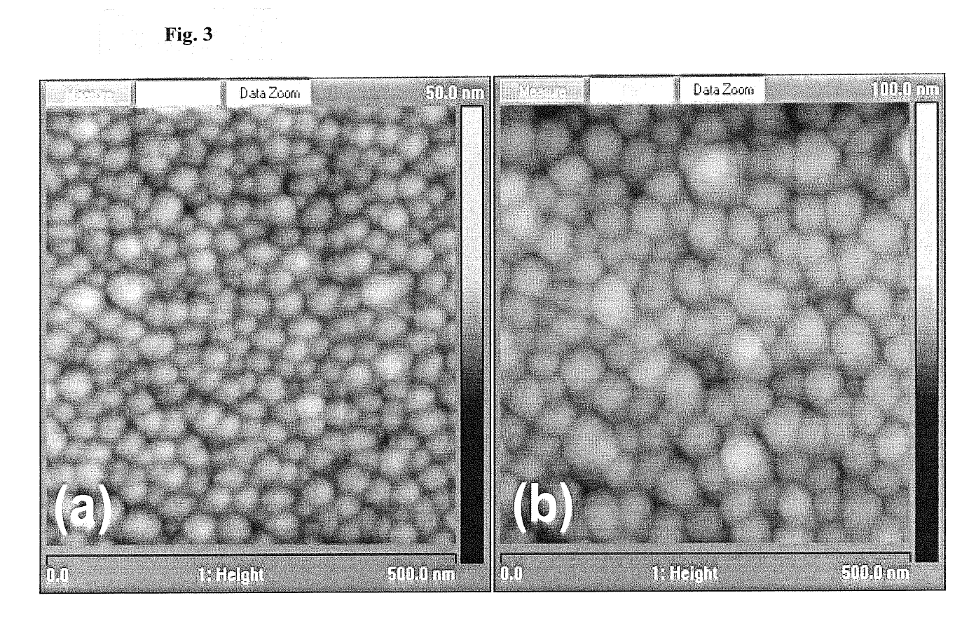

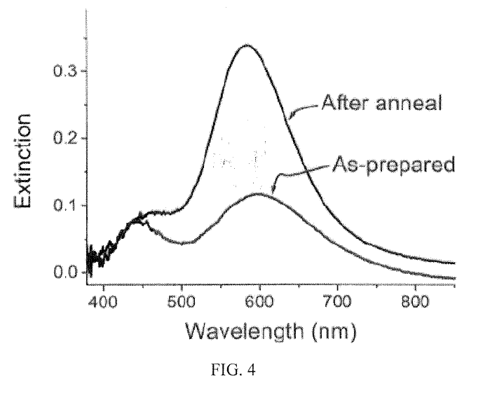

As stated earlier, the annealing of the nanoparticle monolayers leads to a dramatic reduction of the intrinsic hybrid plasmon damping. FIG. 3 depicts the restructuring of Ag nanoparticles after the annealing. In the present embodiment, this annealing step was carried out on a hot plate at 300° C. for 5 minutes. Sintering of the nanoparticles has occurred leading to average particle size increasing from 28 to 52 nm. In other embodiments, different times of temperatures may be sufficient. A before and after anneal comparison of the hybrid plasmon extinction is shown in FIG. 4. The optical extinction peak located at 590 nm is assigned to hybrid plasmon resonance. The peak located at shorter wavelength belongs to the regular dipolar plasmon resonance mode. The redshift of this peak with annealing is consistent with the particle growth. Upon annealing, the hybrid band is seen to increase dramatically. The damping factor or full width at half maximum of the hybrid peak is found to narrow from 0.72 to 0.48 eV upon annealing. This pronounced narrowing of the hybrid band can only be explained by reduction in plasmon damping.

The reduction of intrinsic damping can result from a decrease in dephasing (radiative damping), or decrease in electron scattering (non-radiative damping), or both. At first, the particle size increase is contrary to the decrease in radiative damping. This is because, the retardation effects (leading to radiative damping) in single metal nanoparticles increase with increase in size. However, it is possible that, the restructuring in interacting nanoparticle systems (i.e., increase in) may account for decrease in the overall radiative damping of hybrid plasmon mode despite an increase in average particle size. This decrease in radiative damping can take place due to the establishment of optimum particle size and center to center distance. Further, decrease in damping can also be due to decreased electron scattering by rectification of structural defects like dislocations and grain boundaries at higher thermal energy defects.

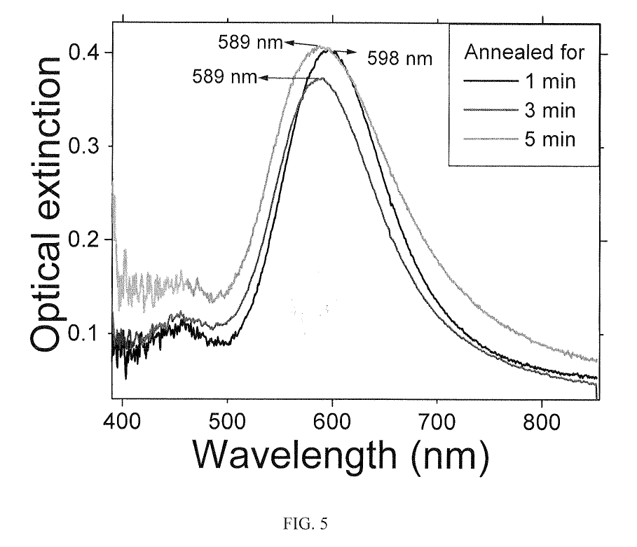

FIG. 5 shows the optical extinction of the silver nanoparticles annealed for different time intervals at 300° C. After several replications, it is concluded that, annealing above 1 min is not beneficial. Rather, longer annealing times lead to increase in hybrid plasmon damping, likely due to oxidation of the silver surface (non-radiative damping). On the other hand, annealing at 250° C. for 5 min is found to similar damping factor as annealing at 300° C. for 1 min.

7. Example Application to Trace-level Mercury Sensing

Mercury is a severe neurotoxin whose contamination in the environment has jumped threefold since the beginning of the industrial revolution. Coal-fired power plants are the major emitters of mercury, which enters the food chain via the air and water. With the emergence of surging economies around the globe and the resulting demands on oil supplies, coal usage and, therefore, the potential for further mercury contamination are increasing. To monitor mercury levels, quick reliable means of both elemental and ionic mercury detection are needed.

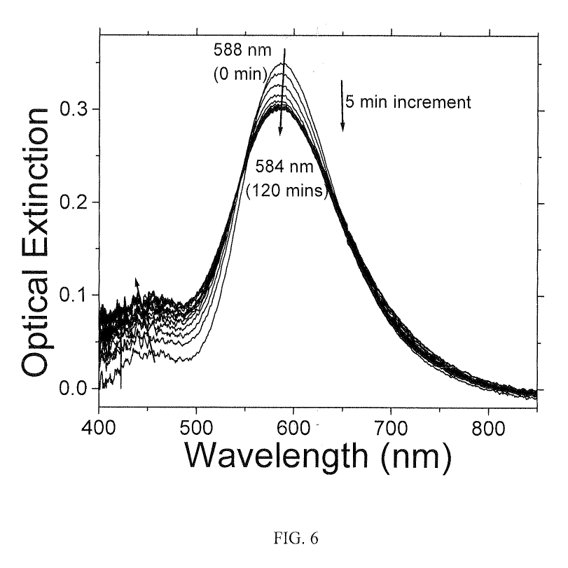

The present disclosure demonstrates the testing of fabricated hybrid plasmon damping sensors against mercury vapor. FIG. 6 shows the time series optical extinction spectra of a silver nanoparticle monolayer measured for every 5 minutes (0 to 120 minutes) after the introduction of 1 g mercury bubble in the optical cell. It is concluded from the FIG., that the extinction of the hybrid plasmon mode decreases (an 8% decrease is recorded in 30 s after mercury exposure) and regular dipolar plasmon mode blue shifts with time. The vaporized mercury atoms adsorb on the silver nanoparticles and adsorption of mercury leads to increase in damping. The increase in damping in turn decreases the extinction of hybrid plasmon mode. Silver being more electronegative than mercury, electrons are donated by the mercury to the silver nanoparticle; increasing total number of conduction electrons Nₑ in silver. This increase in number of conduction electrons increases bulk frequency ωₚ accounting for the blue shift of dipolar plasmon mode.

The measured optical extinction of the silver nanoparticles were fitted to Lorentzians using a computational algorithm. H, extinction peak height, (corrected height after background subtraction) and Γ, damping factor were obtained by least squares curve fitting technique. Subsequently, Equation 4.8 was exploited to compute the number of mercury adsorbates (ΔN_{Hg}):

$$Δ {N}_{Hg} = {N}_{eo} ( \frac{H Γ}{{H}_{o} {Γ}_{o}} - 1 ) \qquad ( 4.8 )$$

H, Γ, and normalized number of adsorbates

$$\frac{Δ N}{{N}_{o}}$$

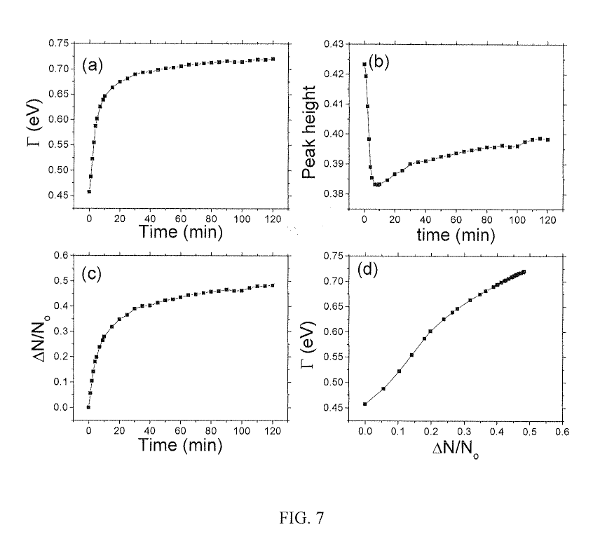

were plotted as a function of time as shown in FIG. 7.

It is evident from the FIG. 7A and FIG. 7C that the damping and number of adsorbates increases initially and reaches a saturation value. These two parameters follow Langmuir adsorption isotherm pattern:

$$Δ N = N \frac{{K}_{ad}}{{K}_{ad} - {K}_{de}} ( 1 - {e}^{- ( {K}_{ad} + {K}_{de} ) t} ) ,$$

where K_{ad} and K_{de} are adsorption and desorption rate coefficients. FIG. 7B depicts the trend of the peak height due to mercury adsorption; it decreases initially (for first 10 minutes), after reaching minimum. It increases steadily with time. Later, it reaches saturation, after filling all the adsorption sites. The intensity of the plasmon extinction peak can vary due to (from Equation 4.5): 1) electron transfer between the adsorbate and the nanoparticle (numerator); and 2) increased damping due to the increased scattering of the electrons by the adsorbate (denominator). From FIG. 7B it is found that the role of the increasing damping factor increases and becomes dominant with the adsorption of mercury atoms on the silver nanoparticles. Further, Nₒ+ΔN also increases due to transfer of electrons from mercury atoms to silver nanoparticles, increasing the conduction electrons in silver nanoparticles. Each adsorbed mercury atom contributes an electron to the silver nanoparticle, so ΔN is linear with the number of adsorbed mercury atoms. In addition, if it is assumed the damping rate is linear with the number of adsorbates; i.e. ΔΓ=cΔN then Equation 4.5 becomes

$${σ}_{ext , peak} = k \frac{{N}_{o} + Δ N}{{Γ}_{o} + c Δ N} .$$

Accordingly, σ_{ext,peak} should only either decrease or increase with ΔN which is contrary with the sensor results in FIG. 7. Hence the assumption that damping rate varies linearly with number of adsorbates is invalid and the damping varies sub-linear with number of adsorbates. Aforementioned relation between damping and number of adsorbates is also fortified from FIG. 7D.

8. Example of Detection of Trace-level (25 ppb) H₂S

Fuel cells have attracted broad attention in recent years due to their ability to deliver electric power as long as their fuel is replenished. Unlike conventional rechargeable batteries, fuel cells can provide remote operation for years with no down time with portable light weight fuels like H₂. In the absence of combustion and corrosion and movable parts, fuel cells do not require maintenance. Water being the only product, fuel cells do not pollute the environment, either. However, fuel cells have a downfall due to the impurities in the fuel being utilized. In particular, sulfur-related impurities, H₂S being the most common, are detrimental to the operation of fuel cells. This is because S has high affinity for catalytic electrodes employed in fuel cells, such as nickel. Reformers generate hydrogen by breaking down hydrocarbons through catalytic processes. An integrated system of a fuel reformer and a fuel cell can provide portable and mobile power. However, diesel and jet fuels are heavy fuels that are difficult to reform, especially with their aromatic and organosulfur impurities. As a result, extensive diagnostic equipment is required to monitor reformer hydrogen output purity. Missing any organosulfur in the production stream will degrade the fuel cell's power production, eventually leading to its replacement. A low-cost but high-sensitivity detection technique is needed to continuously monitor the process stream of fuels for sulfur compounds.

The development of reliable fuel cell technology will have tremendous impact towards developing a robust hydrogen economy. To utilize a source of hydrogen from reformers has been a major technical barrier to utilize fuel cells as power sources in the defense applications. An inexpensive and innovative method is needed to continuously monitor the process stream of military logistical fuel (JP8) for sulfur and other compounds which would harm the reformer catalyst and fuel cell operation in order to reduce system costs and logistical footprint.

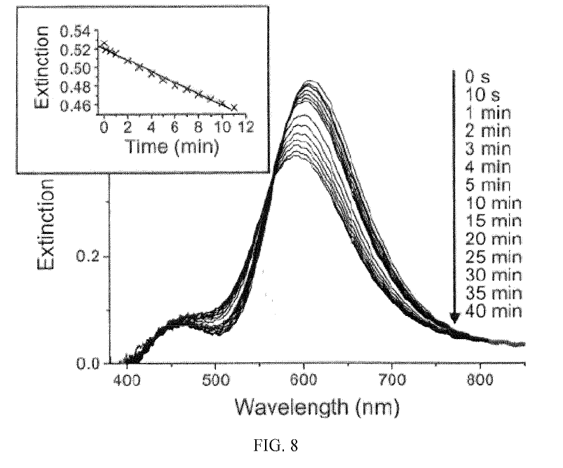

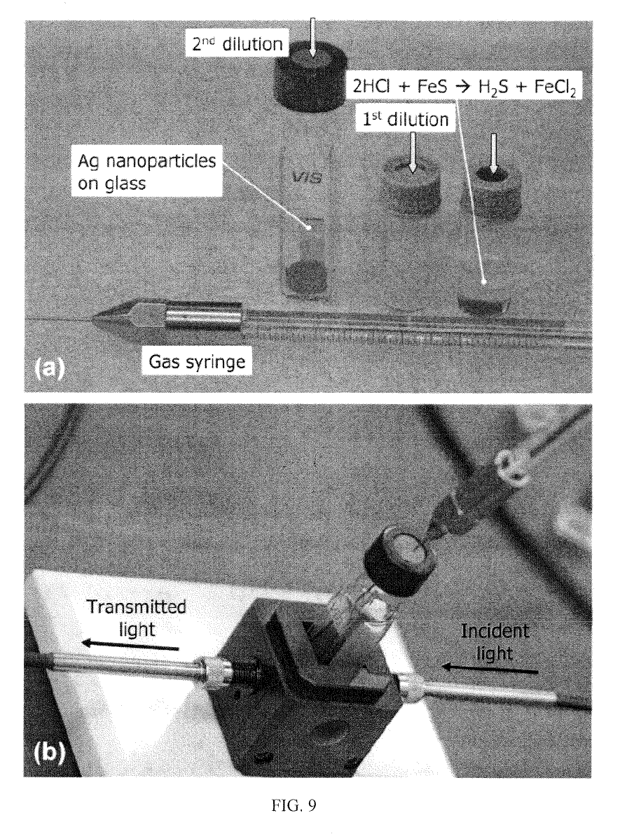

This section of the present disclosure will demonstrate detection of 25 ppb H₂S in 10 s or less exploiting the hybrid plasmon damping as seen in FIG. 8. In this demonstration, H₂S was obtained through the reaction: FeS+2HCl→H₂S+FeCl₂ in a septum sealed vial. The concentration of H₂S accumulating in the vial was adjusted by quantity of FeS. Once, all the FeS reacted, 10 μL of H₂S was removed from the vial by a gas syringe and injected into septum sealed vials for dilution. The nanoparticle coated sample (5 mm×10 mm) was fixed in a 4 mL septum sealed UV-VIS cuvette cell, which was purged with N₂ using syringe needles right after the sample was annealed and fixed in the cell as illustrated in FIG. 9. The time series extinction spectra were captured once H₂S was injected to the cell to a final dilution of 25 ppb as given by FIG. 8. The inset of FIG. 8 plots the peak extinction as a function of time for the initial 11 minutes.

Note that in FIG. 8 the regular plasmon peak (small peak) is not as sensitive to H₂S, while a discernable drop in the hybrid mode was detected in the first 10 seconds or less. Unlike Hg, S shares with or steals electrons from Ag. Therefore, it is a valid argument that a decrease in plasmon peak is also expected from a decrease in electron density. However, the full width at half maximum (FWHM) of the hybrid plasmon peak of FIG. 7 increases from 0.5 eV to 0.55 eV in the first minute of H₂S exposure. This can only be explained by plasmon damping. Detecting 1 ppm H₂S without annealing has been tried, and a very slow response was observed. Therefore, the superior sensing here is attributed to unique hybrid plasmon damping mechanism. In particular, the peak drops the fastest in the first 10 s, when H₂S is injected.

In summary, the LSPR sensor of the present disclosure differentiates itself from others on the basis of at least two unique aspects. First, the sensor utilizes the hybrid plasmon mode (peak) as the sensing probe rather than the regular dipolar plasmon mode (peak). The present disclosure teaches obtaining strong and well-resolved hybrid plasmon resonance peak exploiting its “nanometal-on-semiconductor” approach as well as its “electroless reduction on silicon” nanofabrication technique. The hybrid plasmon resonance is made even better resolved and sensitive to adsorption-induced damping by an annealing step. Second, the LSPR sensor of the present disclosure makes use of a different sensing mechanism. Unlike previous LSPR sensing demonstrations, which monitor frequency shifts due to either refractive index or electron density changes, the present sensor probes two measurables: 1) the hybrid plasmon damping, which is full width at half maximum (FWHM) of the optical extinction peak of the hybrid mode; 2) the intensity of the extinction peak for the hybrid mode. Once these two parameters are recorded, a calculation, also disclosed herein, is performed to precisely quantify the number of electrons gained or lost by the hybrid plasmon mode which in turn quantifies the number of adsorbates and concentration.

The present invention is well adapted to carry out the objectives and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within the spirit of this invention as defined by the claims.

What is claimed is:

1. A method for measuring the concentration of an agent in an environment, comprising:
providing a substrate;
coating the substrate with noble metallic nanoparticles;
exposing the coated substrate to the environment; and
determining the existence of the agent from variation in the hybrid plasmon extinction peak of the metallic nanoparticles.

2. The method of claim 1, further comprising annealing the coated substrate.

3. The method of claim 1, wherein providing a substrate further comprises providing a semiconductor substrate.

4. The method of claim 3, wherein the semiconductor is silicon.

5. The method of claim 1, wherein providing a substrate further comprises providing a silicon on glass covered substrate.

6. The method of claim 1, wherein coating the substrate with noble metallic nanoparticles further comprises immersing a semiconductor film in a metal salt solution.

7. The method of claim 1, wherein annealing the coated substrate further comprises heating the coated substrate to about 300 degrees Celsius for about 1 minute.

8. The method of claim 1, wherein determining the existence of the agent from variation in the hybrid plasmon extinction peak of the metallic nanoparticles further comprises determining a width of a hybrid Plasmon resonance peak.

9. The method of claim 1, wherein determining the existence of the agent from variation in the hybrid plasmon extinction peak of the metallic nanoparticles further comprises determining an intensity of a hybrid Plasmon resonance peak.

10. The method of claim 1, further comprising determining an amount of the agent from the hybrid plasmon extinction peak.

11. The method of claim 1, wherein coating the substrate with noble metallic nanoparticles further comprises providing a monolayer of metallic nanoparticles on the substrate.

12. The method of claim 1, further comprising functionalizing the metallic nanopoparticles to provide selective detection of the agent.

13. A method for measuring an agent in an environment, comprising:
providing a substrate;
coating the substrate with noble metallic nanoparticles;
exposing the coated substrate to the environment; and
determining by spectrophotometry from a variation in a damping factor of the hybrid resonance of the metallic nanoparticles whether the substrate has been exposed to the agent.

14. The method of claim 13, further comprising annealing the coated substrate.

15. The method of claim 13, wherein coating the substrate with noble metallic nanoparticles further comprises providing a monolayer of noble metallic nanoparticles on the substrate.

16. The method of claim 13, further comprising determining a concentration of the agent based on the damping factor.

17. The method of claim 13, wherein providing a substrate further comprises providing a semiconductor film substrate.

18. The method of claim 13, wherein coating the substrate with noble metallic nanoparticles further comprises immersing a silicon film in a metal salt solution.

19. A sensor for measuring concentration of an agent, comprising
a substrate;
a monolayer of noble metallic nanoparticles on the substrate; and
means for detecting the presence and concentration of the agent by measuring variation in the hybrid plasmon extinction peak of the metallic nanoparticles.

20. The sensor of claim 19, wherein:
the substrate is a semiconductor film on glass; and
the monolayer of noble metallic nanoparticles on the substrate is annealed.
